(19) United States
(12) Patent Application Publication
(10) Pub. No.: US 2025/0235551 A1
(43) Pub. Date: Jul. 24, 2025

(54) POROUS MICROCARRIERS FOR THERAPEUTIC LIPID NANOPARTICLES

(71) Applicants: The United States Government as represented by the Department of Veterans Affairs, Washington, DC (US); The Trustees of the University of Pennsylvania, Philadelphia, PA (US)

(72) Inventors: Auhin K. Maparu, Philadelphia, PA (US); Lachlan J. Smith, Philadelphia, PA (US); Michael J. Mitchell, Philadelphia, PA (US); Robert L. Mauck, Philadelphia, PA (US); Zain Siddiqui, Philadelphia, PA (US); Keerthana Iyer, Philadelphia, PA (US)

(21) Appl. No.: 19/030,290

(22) Filed: Jan. 17, 2025

Related U.S. Application Data
(60) Provisional application No. 63/624,592, filed on Jan. 24, 2024.

(51) Int. Cl. A61K 48/00; A61K 9/16; A61K 9/51; A61K 31/7088; A61P 19/02

(52) U.S. Cl. CPC A61K 48/0041; A61K 9/1647; A61K 9/5123; A61K 31/7088; A61P 19/02

(57) ABSTRACT

Porous microcarriers prepared from a poly(lactide-co-glycolide) and a porogen, which are suitable for loading of therapeutic lipid nanoparticles into the pores thereof.

### CROSS-REFERENCE TO RELATED APPLICATIONS

This application claims the benefit of U.S. Provisional Application No. 63/624,592, filed Jan. 24, 2024, the entirety of which is incorporated into this application by reference.

### STATEMENT OF GOVERNMENT SUPPORT

This invention was made with government support under contract number R01AR077435, awarded by the National Institutes of Health and I01RX001321, awarded by the Department of Veteran's Affairs. The government has certain rights in the invention.

### BACKGROUND

Lipid nanoparticles (LNPs) have recently emerged as a promising nanomaterial for delivering nucleic acid-based therapeutics. Nucleic acids are susceptible to degradation due to their exposure to endogenous nucleases naturally present in the body. Lipid nanoparticles not only protect these loaded therapeutics from inherent degradation, they can also be designed for target-specific localization of nucleic acids. However, these nucleic acid-based therapeutics exhibit transient expression following administration which lasts for only a few days. Hence, in order to attain significant therapeutic benefits, multiple doses of lipid nanoparticles are generally required over several weeks to months. Such repeated injection is burdensome for patients and healthcare professionals, often requires multiple patient visits to a clinic, and results in repeated inflammation at the site of injection followed by associated complications in patient health. Developing an appropriate carrier for long-term sustained (>7 days) release of lipid nanoparticles can reduce the frequency of therapeutic administration and is a need for a rapid and successful translation of lipid nanoparticle-based therapeutics.

### SUMMARY

Described are kits, compositions, and treatment methods involving a porous microcarrier prepared from a poly(lactide-co-glycolide) copolymer and a porogen. The porous microcarrier can in some aspects have an average pore size of at least 100 nm. The porous microcarrier can be loaded with a nucleic acid-based therapeutic associated with (e.g., encapsulated by) a lipid nanoparticle.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1A shows an exemplary schematic illustrating the process of loading lipid nanoparticles into the porous microcarriers.

FIG. 1B shows an exemplary schematic illustrating release of lipid nanoparticles from the porous microcarriers.

FIG. 2 shows an exemplary schematic illustrating a fabrication approach for preparing the disclosed porous microcarriers.

FIG. 3 shows a scanning electron microscopy (SEM) image of the fabricated porous microcarriers, depicting their morphology and porous nature. Scale bar: 10 μm.

FIG. 4 shows a confocal laser scanning microscopy (CLSM) image of Nile Red-labeled porous microcarriers, depicting their interconnected porous structure. Scale bar: 10 μm.

FIG. 5 is a bar chart showing the degradation profile of PMCs in PBS at 37° C. over time (n=3). *p<0.05 vs all earlier time points.

FIG. 6A shows plots of loading efficiency of porous microcarriers at varying incubation times for different mRNA concentrations of anionic LNPs (n=3).

FIG. 6B shows plots of loading efficiency of porous microcarriers for anionic and cationic lipid nanoparticles at varying incubation times (n=3). *p<0.05 for anionic vs cationic LNPs.

FIG. 7 shows plots demonstrating the effect of batch-wise variation of PMCs on the loading efficiency values for anionic LNPs (1.25 ng of mRNA/μg of PMCs).

FIG. 8 shows contact angle measurement images for PMCs and anionic LNPs coated glass slide.

FIG. 9A shows CLSM images of bare porous microcarrier (control), which showed no red color. Scale bar: 10 μm.

FIG. 9B shows CLSM images of porous microcarrier loaded with lipid nanoparticles. The lipid nanoparticles (with encapsulated mRNA) were labeled with DiR dye for visualization purposes. Scale bar: 10 μm.

FIG. 10 shows a plot of cumulative release of mRNA-lipid nanoparticles of varying mRNA loading concentration from the porous microcarriers over time (n=3). *p<0.05.

FIG. 11A is a plot showing viability of ATDC5 cells treated with LNPs (n=3). *p<0.05.

FIG. 11B is a plot showing viability of ATDC5 cells treated with PMCs (n=3).

FIG. 11C is a plot showing viability of ATDC5 cells treated with a release formulation containing LNP-loaded PMCs (1.25 ng of mRNA/μg of PMCs) (n=3).

FIG. 12 is a plot showing bioactivity of mRNA (1.25 ng of Luc-mRNA/μg of PMCs) released from PMCs in ATDC5 cells at different time-points (n=3). *p<0.05.

FIG. 13 is a plot showing injectability of NR-labeled PMCs for different needle sizes (n=3).

### DETAILED DESCRIPTION

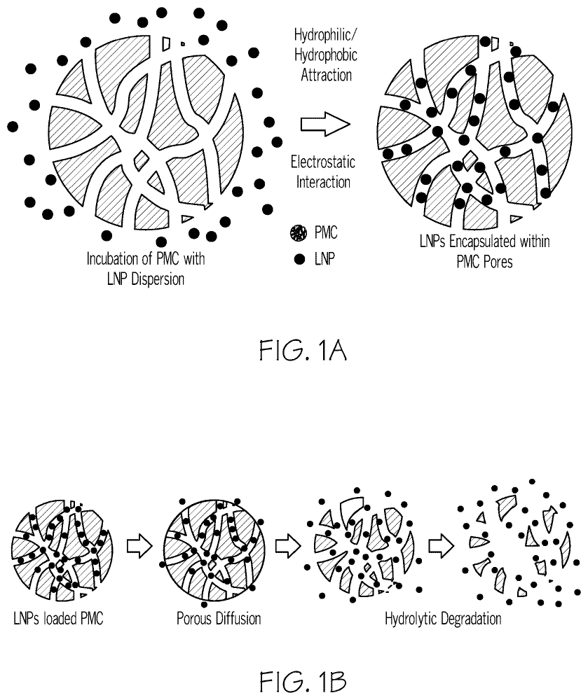

This disclosure relates to poly(lactic-co-glycolic acid) (PLGA) based porous microcarriers for release (including sustained release) of therapeutic lipid nanoparticles associated with a therapeutic agent such as a nucleic acid-based therapeutic. PLGA has good biocompatibility and biodegradability which can be controlled by changing the proportion of lactic and glycolic acid. For example, PLGA microparticles can be made porous using a suitable porogen, and the porosity can be controlled by tuning the amount of porogen. Such porous microcarriers can be incubated with a dispersion of therapeutic lipid nanoparticles (FIG. 1A) to thereby load the lipid nanoparticles within the pores of porous microcarrier via electrostatic interaction or hydrophilic/hydrophobic attraction. Once dispersed in a tissue or biofluid, the combined cargo can exhibit sustained release of the lipid nanoparticles via porous diffusion followed by hydrolytic degradation of the PLGA over time (FIG. 1B).

### I. Porous Microcarrier (PMC)

a. Poly(lactide-co-glycolide)

The microporous carrier generally includes a porous poly(lactide-co-glycolide) (PLGA), which is prepared through the use of a porogen with a base PLGA. In some aspects, the porogen used to prepare the carrier is removed during the fabrication stage to provide the microporous carrier. In one aspect, the PLGA has the following structure:

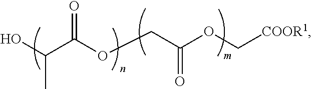

where R¹ is hydrogen (acid terminated PLGA) or C1-C4 alkyl (ester terminated PLGA), and n and m are independently integers of two or greater. In one aspect, R¹ is hydrogen, methyl, ethyl, propyl, or butyl. The terminal functional group (acid or ester) of the PLGA can influence the hydrophilic/hydrophobic interaction of the porous microcarrier with the lipid nanoparticle since an acid terminated PLGA is more hydrophilic than an ester terminated PLGA. Accordingly, release characteristics can be tuned by varying the functional group at R¹. Any stereochemistry of the methyl group on the lactide monomer is contemplated. The lactide in the PLGA may be D,L-lactide or D,D-lactide. The PLGA can have any suitable molecular weight. In one aspect, the molecular weight of the PLGA is 2,000-100,000 g/mol, e.g., 8,000-100,000 g/mol, or 8,000-50,000 g/mol.

Ratios of lactide to glycolide in the PLGA can affect loading, release, and other characteristics via differing hydrophilicity. For example, a lactide to glycolide ratio of 50:50 possesses higher hydrophilicity and exhibits a faster degradation profile as compared to a PLGA with a lactide:glycolide ratio of 85:15. Accordingly, a variety of lactide-glycolide ratios are contemplated, including without limitation those listed in the table below.

| Lactide (%) | Glycolide (%) |
|---|---|
| 95 | 5 |
| 90 | 10 |
| 85 | 15 |
| 80 | 20 |
| 75 | 25 |
| 70 | 30 |
| 65 | 35 |
| 60 | 40 |
| 55 | 45 |
| 50 | 50 |
| 45 | 55 |
| 40 | 60 |
| 35 | 65 |
| 30 | 70 |
| 25 | 75 |
| 20 | 80 |
| 15 | 85 |
| 10 | 90 |
| 5 | 95 |

b. Porogen

In general, the porogen can be any biodegradable or biocompatible material capable of forming pores within the PLGA carrier that are sufficiently large enough to facilitate loading of the lipid nanoparticles. In some aspects, the lipid nanoparticles have a size between 50-200 nm, and thus pore sizes should be sufficiently large enough to allow loading of lipid nanoparticles having this size range.

In one aspect, the porogen can be a material capable of forming average pore sizes of at least 100 nm in the PLGA carrier. In one aspect, the porogen can be a material capable of forming average pore sizes of at least 0.5 μm in the PLGA carrier. In a further aspect, the porogen can be a material capable of forming average pore sizes of 0.5-5 μm in the PLGA carrier. In a further aspect, the porogen can be a material capable of forming average pore sizes of 0.5-3 μm in the PLGA carrier. In a further aspect, the porogen can be a material capable of forming average pore sizes of 0.5-2.5 μm in the PLGA carrier. In a further aspect, the porogen can be a material capable of forming average pore sizes of 0.5-2 μm in the PLGA carrier. In some aspects, the porosity of the porous PLGA carrier is within these ranges, i.e., 0.5-5 μm, 0.5-3 μm, 0.5-2.5 μm, or 0.5-2 μm.

Non-limiting examples of suitable porogens include carboxymethylcellulose (CMC), gelatin, collagen, poloxamers or PLURONIC type porogens (i.e., copolymers of polyethylene oxide and polypropylene oxide, such as triblock copolymers), hyaluronic acid, alginate, chitosan, fibrin, agarose, poly(acrylic acid), poly(vinyl alcohol), poly(vinyl phosphonic acid), poly(glutamic acid), poly(ethylene glycol), poly(ethylene oxide), poly(phosphazene), oligo (poly(ethylene glycol) fumarate), poly(N-isopropyl acrylamide), and poly(hydroxyethyl methacrylate), among others.

In one aspect, the porogen is a copolymer, for example, a triblock copolymer, of polyethylene oxide (PEO) and polypropylene oxide (PPO). For instance, the porogen can be a triblock copolymer comprising PEO-PPO-PEO, corresponding to the following structure:

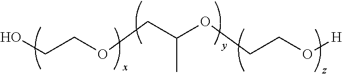

wherein x, y, and z are independently integers ranging from 2 to 200. The ratio of each monomer in such a PEO-PPO-PEO triblock copolymer can affect the hydrophilic/lipophilic balance (HLB) value and water extractability, which can in turn affect pore size, release characteristics of a loaded lipid nanoparticle, among other variables. In one aspect, variables x and z are roughly the same integer, or within 10% of one another, and variable y, which corresponds to the hydrophobic block of the polymer, is independently the same or a different integer.

In one aspect, both of x and z range from 2-140, and y ranges from 10-80. In a further aspect, both of x and z range from 10-140, and y ranges from 10-80. In a further aspect, both of x and z range from 15-140, and y ranges from 10-80. In a further aspect, both of x and z range from 20-140, and y ranges from 10-80. In a further aspect, both of x and z range from 30-140, and y ranges from 10-80. In a further aspect, both of x and z range from 80-140, and y ranges from 10-80. In a further aspect, both of x and z range from 90-140, and y ranges from 10-80. In one specific aspect, both of x and z range from 90-100, and y ranges from 50-70. The degree of polymerization of each monomer can be determined by methods known in the art. Although the end groups in the above structure are hydroxyl, other end groups such as functionalized end groups are contemplated. Other types of micelle-forming copolymers are also contemplated.

In a specific aspect, the PEO-PPO-PEO triblock copolymer can be a type of PLURONIC, a series of commercially available polymers. Non-limiting examples include those in the table below, corresponding to the following x, y, and z integer values.

| PLURONIC Type | z | y | z |
|---|---|---|---|
| F68 | 80-85 | 25-35 | 80-85 |
| F108 | 130-140 | 50-55 | 130-140 |
| F127 | 90-100 | 50-70 | 90-100 |
| L35 | 10-15 | 10-20 | 10-15 |
| L44 | 10-15 | 15-25 | 10-15 |
| L64 | 10-15 | 25-35 | 10-15 |
| P85 | 20-30 | 30-45 | 20-30 |
| P105 | 30-35 | 40-60 | 30-35 |
| L61 | 2-5 | 25-35 | 2-5 |
| P123 | 15-25 | 50-70 | 15-25 |
| L121 | 2-10 | 50-70 | 2-10 |

In one aspect, the PLGA porous microcarriers can be prepared by emulsifying the PLGA polymer with the porogen in a suitable organic solvent such as dichloromethane. The weight ratio of the PLGA and the porogen can vary depending on the desired porosity characteristics. In some aspects, in the emulsification step, the PLGA polymer and the porogen can be present at weight ratios of 2:3 to 1:9 (PLGA:porogen). The organic mixture or solution of PLGA and the porogen can be emulsified for instance with the assistance of a polymer such as polyvinyl alcohol (PVA) using a mixing device such as a homogenizer. After this process, organic solvent can be removed, which also can remove some or all of the porogen, leaving the porous PLGA carrier. Accordingly, in some aspects, the PLGA and the porogen can have different solubility profiles to aid in the removal of the porogen once pores are created, if desired.

The size of the porous PLGA-based microcarrier can vary. In general, porous microcarriers having a size less than 10 μm can be captured by macrophages. On the other hand, if the porous microcarrier is too large (>100 μm), the microcarrier can cause difficulty during syringe injection. As a result, in one aspect, the porous PLGA-based microcarrier can have an average particle size of 10-100 μm. In a further aspect, the porous PLGA-based microcarrier can have an average particle size of 10-80 μm, e.g., 10-70 μm, 10-60 μm, 10-50 μm, or 10-40 μm. In a further aspect, the porous PLGA-based microcarrier can have an average particle size of 20-80 μm, e.g., 20-70 μm, 20-60 μm, 20-50 μm, or 20-40 μm. The size of the porous microcarrier can be tuned by varying the homogenization speed (higher speed yields smaller porous microcarriers) of the solution comprising the PLGA and the porogen. In a further aspect, changing the porogen to PLGA ratio can also induce changes in the size of porous microcarrier owing to the density difference between the two components.

### II. Lipid Nanoparticle (LNP)

The lipid nanoparticle can be any particle of any morphology which in general is formulated with cationic or ionizable lipids and typically mixed with an aqueous environment in the presence of the cargo entity described below. For example, a liposome, a lipid complex, a lipoplex, an emulsion, a micelle, a lipidic nanocapsule, a nanosuspension, among others, are within the scope of the lipid nanoparticle. In some aspects, the lipid nanoparticle comprises (a) at least one of a cationic lipid or ionizable lipid (or one or more of both), (b) a structural lipid, (c) a sterol, and optionally (d) a polyethylene glycol (PEG)-lipid conjugate. In addition, as described below, the lipid nanoparticle can be associated with a cargo entity such as a nucleic acid-based therapeutic, which can for example be encapsulated or partially encapsulated within the lipid nanoparticle. The lipid nanoparticle can have any suitable size, which can be tailored based on the composition of the lipid nanoparticle components. In some aspects, the lipid nanoparticle has a size ranging from 20-200 nm.

a. Cationic Lipid

A variety of cationic lipids can be used as part of the lipid nanoparticle. In one aspect, the cationic lipid carries a net positive charge at about physiological pH. In a further aspect, the cationic lipid may be an amino lipid, or a lipid having one or two fatty acid or fatty alkyl chains and an amino head group (including an alkylamino or dialkylamino groups) that may be protonated to form a cationic lipid at physiological pH. Thus, in a composition such as a pharmaceutical composition, the ionizable cationic lipid may be neutral or not yet cationic depending on the pH of the composition.

Non-limiting examples of suitable cationic lipids include N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-diolcoyloxy)propyl)-N,N,N-trimethylammonium chloride and 1,2-Dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-γ-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), 1,2-Dilinoleoyl-3-trimethylaminopropane chloride salt (DLin-TAP-Cl), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino) butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), or any combination thereof.

Other suitable cationic lipids include N,N-distearyl-N,N-dimethylammonium bromide (DDAB), 3P—(N—(N′,N′-dimethylaminoethane)-carbamoyl) cholesterol (DC-Choi), N-(1-(2,3-dioleyloxy) propyl)-N-2-(sperminecarboxamido)ethyl)-N,N-dimethylammonium trifluoracetate (DOSPA), dioctadecylamidoglycyl carboxyspermine (DOGS), 1,2-dilcoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, for example, LIPOFECTIN (including DOTMA and DOPE), and LIPOFECTAMINE (comprising DOSPA and DOPE). In one specific aspect, the lipid nanoparticle comprises 10-50% DOTAP by weight of the lipid nanoparticle, e.g., 10% or 40% DOTAP by molar ratio of the nanoparticle.

b. Ionizable Lipid

The lipid nanoparticle may have a cationic lipid, an ionizable lipid, or one or more of both such lipids. Ionizable lipids are protonated at low pH, which makes them positively charged, but they remain neutral at physiological pH. The pH-sensitivity of ionizable lipids is useful for delivery of a nucleic acid-based therapeutic in vivo because neutral lipids have less interactions with the anionic membranes of blood cells and thus can improve the biocompatibility of the lipid nanoparticles. When trapped in endosomes, where the pH is lower than in the extracellular environment, ionizable lipids are protonated and become positively charged, which may promote membrane destabilization and facilitate endosomal escape of the nanoparticles.

Any suitable ionizable lipid can be used. Non-limiting examples include 1,2-dilinoleyloxy-N,N-dimethyl-3-aminopropane (DLin-DMA), 2,2-dilinoleyl-4-dimethylaminoethyl-[1,3]-dioxolane (DLin-KC2-DMA), (6Z,9Z,28Z,31Z)-heptatriaconta-6,9,28,31-tetraen-19-yl 4-(dimethylamino) butanoate (DLin-MC3-DMA; MC3), (2S)-2,5-bis(3-aminopropylamino)-N-[2-(dioctadecylamino)acetyl]pentanamide (DOGS; Transfectam), N1-[2-((1S)-1-[(3-aminopropyl)amino]-4-[di(3-aminopropyl)amino]butylcarboxamido)ethyl]-3,4-di[oleyloxy]-benzamide (MVL5), DC-Cholesterol, N4-cholesteryl-spermine (GL67), di((Z)-non-2-en-1-yl) 9-((4-(dimethylamino)butanoyl)oxy)heptadecanedioate (L319), heptadecan-9-yl 8-((2-hydroxyethyl)(8-(nonyloxy)-8-oxooctyl)amino)octanoate (Lipid 5), heptadecan-9-yl 8-((2-hydroxyethyl)(6-oxo-6-(undecyloxy)hexyl)amino)octanoate (Lipid H (SM-102)), ((4-hydroxybutyl)azanediyl)bis(hexane-6,1-diyl)bis(2-hexyldecanoate) (ALC-0315)

c. Structural Lipid

A variety of structural lipids can be used. Common structural lipids in lipid nanoparticle compositions are often phospholipids. Suitable phospholipids include for example, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-diolcoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some aspects, the lipid nanoparticle includes DSPC. In further aspects, the lipid nanoparticle includes DOPE. In a further aspect, the lipid nanoparticle includes both DSPC and DOPE.

d. Sterol

Any suitable sterol can be used in the lipid nanoparticle. Suitable non-limiting examples include cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

c. Polyethylene Glycol (PEG)-Lipid Conjugate

Optionally, the lipid nanoparticle can include a PEG-lipid conjugate. A PEGylated lipid may include PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid can be PEG-c-DOMG, PEG-DMG, PEG-DLPE, PEG-DMPE, PEG-DPPC, or a PEG-DSPE lipid.

f. Nucleic Acid-Based Therapeutic

In general, the lipid nanoparticle can be associated with any nucleic acid-based therapeutic. In some aspects, the lipid nanoparticle encapsulates or partially encapsulates the nucleic acid-based therapeutic. In some aspects, the nucleic acid-based therapeutic can be RNA, DNA, mRNA, miRNA, siRNA, pDNA, microRNA, ncRNA, engineered msDNA, engineered RT, guide RNAs, vectors and vector systems, and the like. Non-limiting therapeutics include those used for the treatment of musculoskeletal disorders such as intervertebral disc degeneration (a cause of low back pain), synovial joint degeneration (e.g., due to degenerative or juvenile arthritis, or developmental disorders), mucopolysaccharidoses, and other types of arthritis. These conditions are leading causes of disability in both adults and children, and currently lack effective treatments.

Without being bound by any theory, potential mechanisms of action can include local suppression of inflammation, regeneration of healthy tissue, or replacement of proteins that are deficient due to genetic abnormalities. Combinations of each of these approaches are also possible. The therapeutic can also be employed for sustained release of therapeutic RNA at the site of administration for a wide range of other conditions, both musculoskeletal and otherwise, such as cancer, liver fibrosis, cardiovascular disorders and diseases, skin diseases (e.g. wound healing), and bone fracture healing.

g. Combining Lipid Nanoparticle with Porous Microcarrier

An advantage of the disclosed composition and method is the way in which the lipid nanoparticle is loaded into the porous microcarrier. In general, loading can be accomplished with simple mixing. For example, the porous microcarrier, in a freeze-dried state for instance, can be incubated within an aqueous dispersion (e.g., a dispersion in phosphate buffered saline) containing a suitable amount of the lipid nanoparticles (comprising a loaded amount of nucleic acid-based therapeutic), and mixed on a gentle shaker for a sufficient amount of time. Since the loading of lipid nanoparticles involves simple mixing of porous microcarriers with the lipid nanoparticle dispersion, and is carried out after the fabrication of the porous microcarrier, the nucleic acid-based therapeutic or the lipid nanoparticles are not exposed to any harsh environment (e.g., harmful or toxic substances or heating and cooling cycles of porous microcarrier fabrication steps), and hence will be less prone to degradation.

### III. Pharmaceutical Composition, Kit, and Treatment Methods

The loaded porous microcarriers may be provided as a pharmaceutical composition, or in some aspects, a kit. The kit can comprise the porous microcarrier and the loaded lipid nanoparticle, where the porous microcarrier is separate from or not mixed with the lipid nanoparticle. These two components can be packaged together or separately and can be sold with additional components, such as a syringe for instance, when the method of administration is parental such as a local injection.

Pharmaceutical compositions can be in any suitable form. A pharmaceutical composition may be prepared, packaged, or sold in bulk, as a single unit dose, or as a plurality of single unit doses. The composition may include a pharmaceutically acceptable carrier such as a liquid or solid filler, diluent, excipient, manufacturing aid (e.g., lubricant, talc magnesium, calcium or zinc stearate, or steric acid), or solvent encapsulating material, involved in carrying or transporting the material from one site (e.g., the delivery site) of the body, to another site (e.g., organ, tissue or portion of the body).

In some aspects, the pharmaceutical composition is formulated for delivery to a subject, e.g., for treatment of a particular disorder. Non-limiting routes of administrating the pharmaceutical composition include: topical, subcutaneous, transdermal, intradermal, intralesional, intraarticular, intradiscal, intraperitoneal, intravesical, transmucosal, gingival, intradental, intracochlear, transtympanic, intraorgan, epidural, intrathecal, intramuscular, intravenous, intravascular, intraosseous, periocular, intratumoral, intracerebral, and intracerebroventricular administration.

In some aspects, the pharmaceutical composition is administered locally to a target or diseased site. In some aspects, the pharmaceutical composition is administered to a subject by injection, by means of a catheter, by means of a suppository, or by means of an implant, the implant being of a porous, non-porous, or gelatinous material, including a membrane, such as a silastic membrane, a fiber, or a hydrogel.

In some aspects, the pharmaceutical composition is formulated in accordance with routine procedures as a composition adapted for intravenous or subcutaneous administration to a subject, e.g., a human. In some aspects, the pharmaceutical composition for administration by injection can be a solution in sterile isotonic aqueous buffer. Where necessary, the pharmaceutical can also include a solubilizing agent and a local anesthetic such as lignocaine to case pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

The pharmaceutical composition can be prepared as part of the manufacture of a medicament, and can be used to treat an appropriate disorder. In general, any disease or disorder that can be treated by a nucleic acid-based therapeutic is suitable with respect to the disclosed compositions. In one aspect, the disclosed compositions are useful for sustained release of the lipid nanoparticle into a subject, e.g., 1-10 weeks after administration. The efficacy of some lipid nanoparticles alone is often few days, by contrast. Thus, in some aspects, administration to the subject can be a periodically repeating administration depending on the disorder being treating and the treatment plan. Administration of the composition, for instance, can be once every four days, once every five days, once every seven days, once every two weeks, once every month, once every two months, once every three months, once every four months, once every six months, or once every year.

In some specific aspects, the pharmaceutical composition can be delivered to a subject such as a human via local injection directly to or adjacent to target or diseased tissue. In the case of the intervertebral disc, for example, this can include the nucleus pulposus or annulus fibrosus. In the case of the synovial joint, this can include the intra-articular cavity, or articular cartilage. In some aspects, for intra-discal or intra-articular injections, around 10-2000 μl of the pharmaceutical composition, suspended in a delivery vehicle such as sterile saline or other buffer, or a viscous hydrogel, can be administered using a 18 G-33 G needle. Dose, needle size, and injection volume may be highly dependent on the target tissue, age of the patient, type of nucleic acid-based therapeutic being delivered, and desired lipid nanoparticle release duration. In one aspect, to load the lipid nanoparticles, they can be mixed with the porous microcarrier on a shaker for 1-24 hours before administration. LNP loading, and subsequent release following administration will be optimized by varying PMC (PLGA) composition, size and porosity, and can be tuned depending on the application.

### Examples

The following examples further illustrate this disclosure. The scope of the disclosure and claims is not limited by the scope of the following examples.

### I. Fabrication Methods

a. PMC Fabrication

Bare PMCs were fabricated as follows. About 300 mg of PLGA and 700 mg of PLURONIC F127 were dissolved in 3 ml of dichloromethane (DCM) by stirring overnight. The polymer solution was then emulsified in 100 ml of ice-cold aqueous PVA solution (0.5 wt %) using a homogenizer at 3500 rpm for 2 min. The solvent (DCM) was removed via magnetic stirring of the emulsion at 40° C. for 6 h inside a hood. The resulting hardened PMC dispersion was then centrifuged, washed several times with deionized water and finally freeze-dried for long-term storage. For preparing Nile red (NR) labeled PMCs, 3 mg of NR was added into the PLGA solution along with the other ingredients (PLURONIC F127 and DCM) and the subsequent steps were followed as above.

b. mRNA-Encapsulated LNP (mRNA-LNPs) Fabrication

Green fluorescent protein (GFP) or luciferase encoding mRNA (GFP-mRNA or Luc-mRNA) was encapsulated within LNPs. Various LNP formulations were optimized based on transfection in musculoskeletal cells (e.g. cells from cartilage and intervertebral disc). Finally, LNP formulations containing 10% 1,2-diolcoyl-3-trimethylammonium propane (“DOTAP”) and 40% DOTAP (a permanently cationic lipid) were selected for the present Examples.

c. Loading and Release of mRNA-LNPs from PMCs

The loading and release of LNPs were quantified by measuring mRNA content in the LNPs. For loading experiments, 100 μg of freeze-dried PMCs were incubated with 500 μl of LNPs dispersion in phosphate buffered saline (PBS) on a gentle shaker. Five concentrations of mRNA (0.5-10 ng/μg of PMCs) were tested for the loading experiments. After the desired period of incubation, the resulting dispersion was filtered using 0.45 μm syringe filter and the filtrate was collected for measuring the mRNA concentration in LNPs using Triton X-100 and RiboGreen assay kit, following standard protocol. Loading efficiency of the PMCs was calculated as (X−Y)/X*100 where X and Y are the measured mRNA content in the filtrate at the initial stage (denoting total LNPs) and after the desired incubation period (denoting unloaded LNPs). Release of LNPs into PBS at time points up to 28 days was examined at 37° C. under gentle agitation. For release experiments, the LNPs/PMCs (1.25-10 ng of mRNA/μg of PMCs) dispersion was collected at predetermined time points, filtered and then the filtrate was analyzed for determining the mRNA amount (encapsulated within LNPs) released in PBS. All loading and release experiments were performed in triplicate. Statistical differences in LNP loading and release over time were established using two-way ANOVA and one-way ANOVA with Tukey's test, respectively.

d. In Vitro Evaluation of Cytotoxicity and Bioactivity:

5,000 cells were seeded in monolayer in each well of white walled 96 well plate. The cells were treated with varying amount of LNPs, PMCs (equivalent to 1.25 ng of mRNA/μg of PMCs) and release formulation (containing LNP-loaded PMCs after incubation for 35 days) for 24 h. The viability of immortalized ATDC5 cells was measured using CellTiter-Glo assay. Sustained release bioactivity of mRNA at 17, 25 and 28 days was assessed by incubating the Luc-mRNA-LNPs loaded PMC formulation (1.25 ng of mRNA/μg of PMCs) with ATDC5 cells in monolayer for 24 h and measuring luciferase activity relative to untreated cells. Statistical differences in cytotoxicity and bioactivity measurements were established using one-way ANOVA with Tukey's test.

e. Ex Vivo and In Vivo Evaluation for Retention of PMCs:

The injectability of the NR-labeled PMCs was evaluated by determining the fluorescence intensity of the particles after injection through needles of increasingly smaller gauges (23 G, 25 G, 27 G and 30 G) and normalizing the values relative to pipette dispensing. An increase or decrease in fluorescence intensity would indicate rupture or clogging of PMCs within the needle channel, respectively. Statistical differences in fluorescence intensity values were determined using one-way ANOVA with Tukey's test. For ex vivo study, 50 μl of fluorescent, NR-labeled PMCs (1 μg/μl concentration) were injected using a 25 G needle into the nucleus pulposus region of postmortem-isolated healthy adult porcine lumbar discs and then imaged for NR fluorescence signal using an IVIS instrument at time points up to 28 days. As a control, saline injection was performed in another disc. For in vivo study, 100 μl of NR-labeled PMCs (1 μg/μl concentration) were injected using 25 G needle into the right canine knee joint (1 month of age). Saline injection was performed in the left knee joint as a control. Animals were euthanized at time points up to 28 days. The joints were dissected, and the individual tissues were imaged using the IVIS instrument for detection of fluorescence from NR.

### II. Results

a. PMC Fabrication Strategy

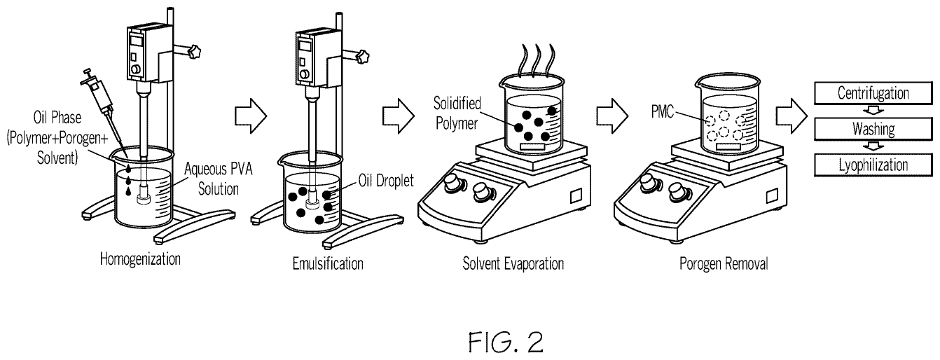

PLGA based PMCs were prepared by dissolving the polymer (PLGA) in its solvent (dichloromethane or DCM) along with a suitable porogen, followed by homogenization of the mixture in water (containing a surfactant as emulsifier) (FIG. 2). The emulsification process produces oil droplets which are dispersed stably in water using the emulsifier (polyvinyl alcohol or PVA).

In the next step, the solvent (DCM) is removed via gentle heating with magnetic stirring. As a result, PLGA gets solidified, but still remains dispersed in the aqueous phase using the emulsifier. The porogen impregnated within the PLGA matrix comes in contact with the outer aqueous phase and eventually leaches out to form pores within the PLGA microparticles. The resulting dispersion is then further centrifuged to collect the PMCs, washed several times with water to remove the excess reagents (including porogen) and finally freeze-dried for long term storage.

b. Process Parameters

An aspect of the described approach is the formation of interconnected pores within the PLGA microparticles. As the pores are formed only when the external aqueous phase reaches the porogen domain of the PLGA matrix, the interconnectivity of the pores is ensured. Hence, even the innermost pores can be accessible for the loading of LNPs, resulting in improved loading capacity of the PMCs. The pore density (number of pores per unit area) of PMCs can be increased by increasing the porogen to PLGA (w/w) ratio. Higher pore density leads to higher loading efficiency, faster degradation of the PMCs (due to availability of higher pore surface area for interaction with water), and rapid release of the LNPs.

The choice of porogen is also a factor to consider. The porogen can possess high hydrophilic/lipophilic balance (HLB) value and water extractability for easy removal from the PLGA domain to the surrounding aqueous phase. PLURONICS are useful since they meet all these criteria. The pore size of the PMCs is also determined by the type of porogen. For example, PLURONIC F127, when used as a porogen, creates larger pores than PLURONIC F68, possibly due to the larger size of the former.

The size of the PMCs can also be taken into consideration while designing PMCs for sustained release applications. PMCs of size lesser than 10 μm are readily captured by macrophages whereas too large (>100 μm) PMCs can cause difficulty during syringe injection. In the described methodology, the size of the PMCs can be easily tuned by varying the homogenization speed (higher speed yields smaller PMCs). Changing the porogen/polymer ratio also induces changes in the size of PMCs owing to the density difference between them.

The composition of PLGA also plays a role in the loading and release of LNPs from PMCs. For example, the terminal functional group (acid/ester) of PLGA influences the hydrophilic/hydrophobic interaction of PMCs with the LNPs since acid terminated PLGA is more hydrophilic than the ester terminated polymers. The ratio of lactic acid (hydrophobic) to glycolic acid (hydrophilic) in PLGA determines the overall hydrophilicity/hydrophobicity of PLGA and the degradation behavior of PMCs. For example, PLGA with a lactic acid to glycolic acid ratio of 50:50 possesses higher hydrophilicity and faster degradation profile than PLGA with the ratio of 85:15.

c. Characterization of PMCs

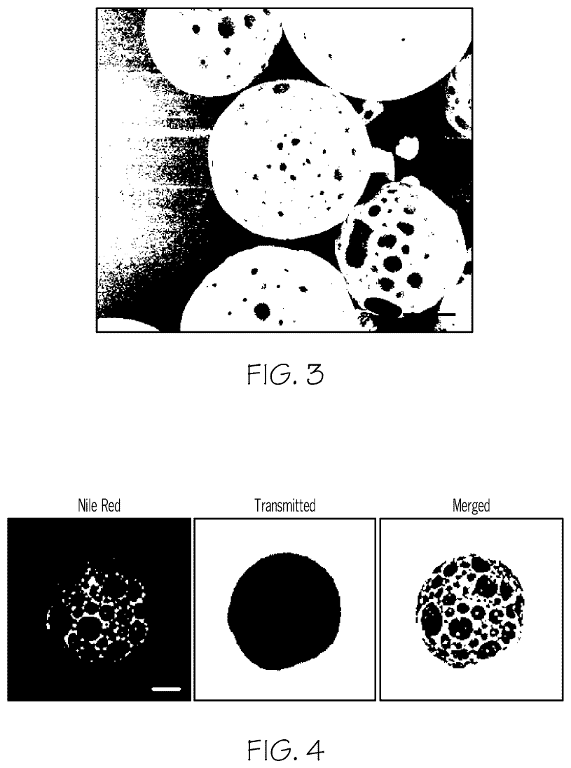

Scanning electron microscopy (SEM) analysis revealed formation of PMCs (FIG. 3) having an average size of 30.35±4.54 μm and a pore size of 1.26±0.76 μm (measured from 10 different images of PMCs). Since the typical size of LNPs is around 100 nm, the pores of the PMCs are large enough to accommodate LNPs without much resistance during loading and release. The size of the PMCs is within the desirable range of 10-100 μm.

In order to visualize PMCs under confocal laser scanning microscopy (CLSM), a fluorescent dye NR was incorporated within the PLGA matrix. As shown in FIG. 4, the fabricated PMCs are highly porous in nature. The interconnectivity of the pores is visible which can aid in the permeation of LNPs even to the innermost section of the PMCs.

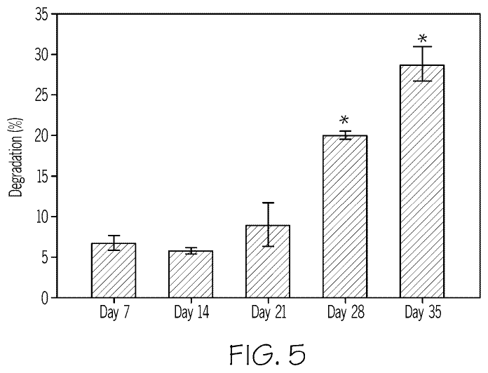

The degradation behavior of the fabricated PMCs was evaluated by dispersing them in PBS and keeping the dispersion inside an incubator maintained at 37° C. to mimic the physiological environment. The weight of the PMCs was measured at different time intervals to determine the degradation (%) with respect to initial weight. As shown in FIG. 5, the PMCs exhibited sustained degradation over time for at least 35 days, further indicating their potential for sustained release of loaded cargo. Notably, the degradation properties can be tuned by varying the PLGA composition.

d. Fabrication and Characterization of mRNA-LNPs

LNPs were fabricated by combining an aqueous phase containing GFP-mRNA with an ethanol phase containing the desired lipids via chaotic mixing using a microfluidic mixer. Two types of LNPs were formulated by varying the proportion of the cationic lipid DOTAP: anionic LNPs (using 10% DOTAP) and cationic LNPs (using 40% DOTAP). The size and zeta potential values of the fabricated LNPs are measured based on dynamic light scattering (DLS) and electrophoretic light scattering (ELS), respectively. The details are summarized in Table 1. The size and zeta potential of the LNPs slightly increase with an increase in DOTAP composition.

TABLE 1

| Type of LNP | DOTAP (%) | Size (nm) | Zeta Potential (mV) |
|---|---|---|---|
| Anionic | 10 | 69.53 ± 28.33 | −5.00 ± 2.41 |
| Cationic | 40 | 111.50 ± 64.91 | 6.05 ± 0.87 |

e. Loading and Release of mRNA-LNPs from PMCs

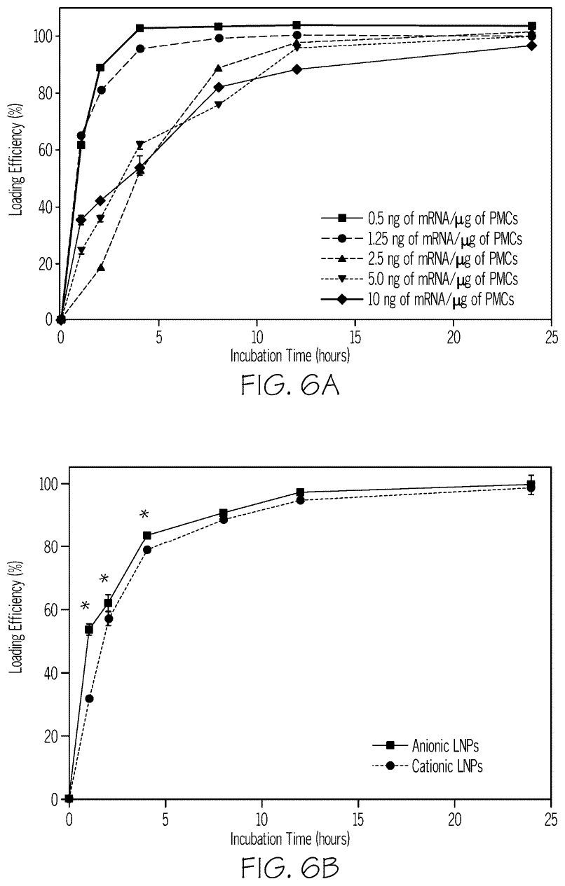

The anionic and cationic LNPs were incubated with bare PMCs to facilitate the loading process. Anionic LNPs were employed for standardization of the loading concentration of mRNA in LNPs. As shown in FIG. 6A, the mRNA concentration of 0.5-1.25 ng/μg of PMCs exhibited faster loading than 2.5-10 ng/μg of PMCs. Additionally, since the mRNA loading amount of 1.25 ng/μg of PMCs was higher than 0.5 ng/μg of PMCs, this concentration (1.25 ng of mRNA/μg of PMCs) was employed for further experiments with anionic as well as cationic LNPs.

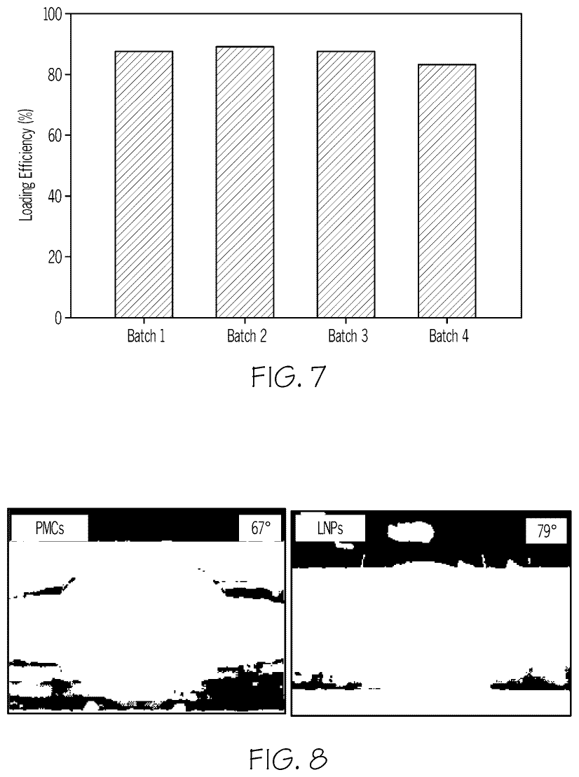

When anionic LNPs (1.25 ng of mRNA/μg of PMCs) were incubated with four different batches of PMCs for 4 h, similar loading efficiency values were observed (FIG. 7). This advocates for the robust fabrication process of the PMCS which is reflected by the batch-wise consistency of the loading efficiency values.

The surface wettability of the PMCs and LNPs were determined by developing a thin film of LNPs and PMCs on a glass slide and then measuring water contact angle on the coated section of the glass slide. As shown in FIG. 8, the water contact angle values of both PMCs and LNPs are within the range of adhesive contact angle (i.e., 65-80°). Therefore, LNPs would experience hydrophilic attractive forces when at closer proximity with the PMCs. Since bare PMCs are slightly anionic in nature (zeta potential: −7.47±0.78 mV), the conjugation with anionic LNPs was mediated only through hydrophilic attraction whereas cationic LNPs were adsorbed more favorably on the PMC pores, owing to the additional electrostatic attractive forces acting between them. As a result, the loading efficiency values were consistently higher for the cationic LNPs than the anionic LNPs for all the time points considered (FIG. 6B).

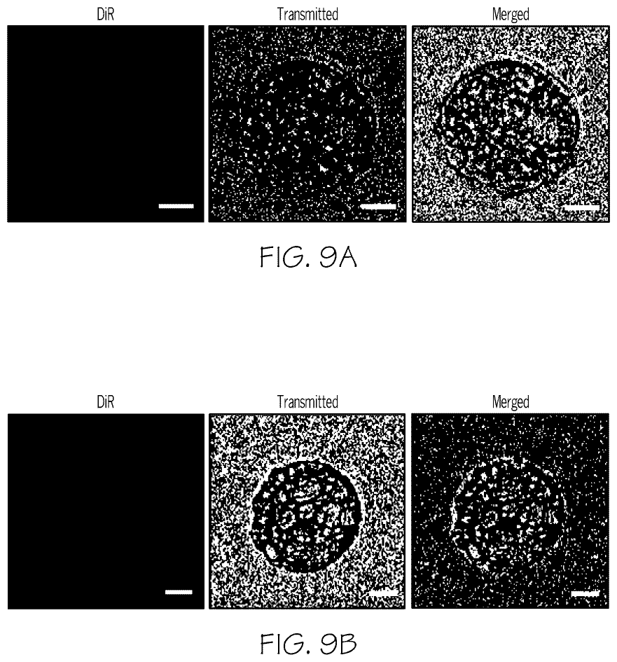

For visualization of the loading process under confocal microscopy, the LNPs were labeled with a fluorescent dye, DiR. As shown in FIG. 9A, bare (unloaded) PMCs did not show any fluorescence in the NIR spectrum (for DiR, λₑₓ=750 nm and λₑₘ=780 nm). When PMCs were loaded with anionic LNPs, clear fluorescence signal was detected throughout the PMCs (FIG. 9B). This experiment confirms successful loading of LNPs throughout the porous matrix of the fabricated PMCs, even when anionic LNPs were used with the standardized concentration of mRNA (1.25 ng of mRNA/μg of PMCs).

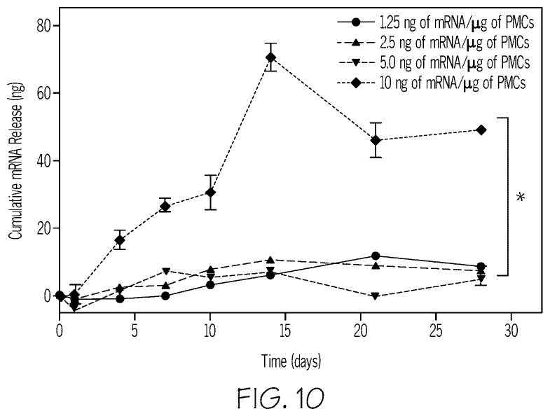

The release profiles of anionic LNPs (1.25-10 ng of mRNA/μg of PMCs) from PMCs is depicted in FIG. 10. Negligible release (undetected using RiboGreen Assay) at the initial time points indicates excellent loading capability of the PMCs. The release kinetics confirmed sustained mRNA release over 28 days and were similar for most loading concentrations, except for 10 ng of mRNA/μg of PMCs, which showed significantly higher release over the first 14 days followed by slight attenuation, possibly due to progressive degradation of accumulated mRNA.

f. In Vitro Evaluation of Cytotoxicity and Bioactivity:

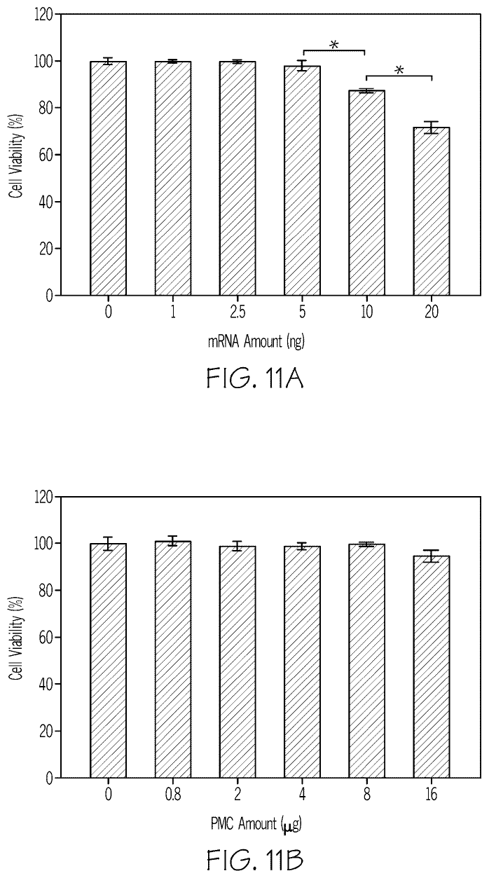

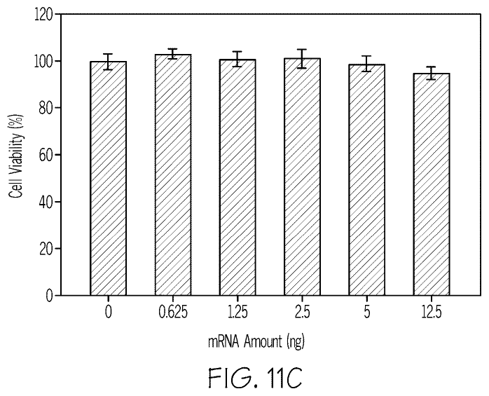

The cytotoxicity potential of the LNPs, PMCs and release formulation (LNPs loaded PMCs after incubation for 35 days) were evaluated in chondrogenic ATDC5 cells. As shown in FIGS. 11A-11C, the LNPs exhibited mild reduction of cell viability at higher dosages, whereas the PMCs and the release formulation did not induce any cytotoxicity to the cells at any dose tested. This affirms that the sustained exposure of LNPs (released from PMCs) to the cells does not have any cytotoxic potential.

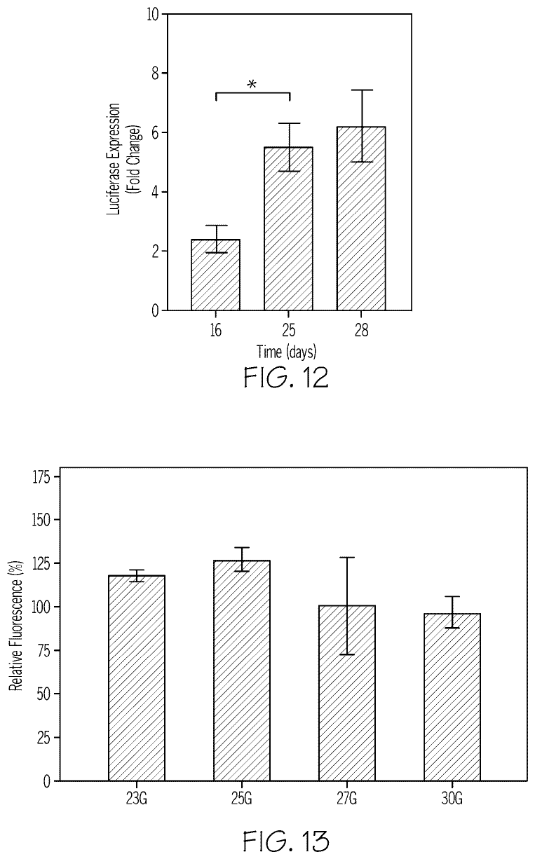

To evaluate the bioactivity of the released mRNA, a luciferase assay was used. Luc-mRNA was encapsulated within LNPs before loading within PMCs. When the cells were exposed to the release formulation (containing 1.25 ng of Luc-mRNA/μg of PMCs) at various time points, luciferase expression was observed in a time-dependent manner (FIG. 12). This indicates that the therapeutic cargo (mRNA encapsulated within LNPs) was able to retain its therapeutic potential when loaded within its carrier (PMCs). The increase in luciferase expression over time further supports the sustained release potential of the fabricated PMCs.

g. Ex Vivo and In Vivo Evaluation for Retention of PMCs:

Rapid clearance of therapeutics from injection site has been a pressing challenge for synovial joints and intervertebral disc. Therefore, retention of PMCs was tested after injection in an ex vivo porcine disc organ culture model and in vivo in canine joints. NR-labeled PMCs were used for easy visualization using fluorescence under IVIS instrument. Prior to injection, the injectability of the NR-labeled PMCs was tested using different needle sizes. Among the tested needle sizes, no significant differences in relative fluorescence (normalized to pipette dispensing) from the NR-labeled PMCs were observed, confirming that there was no needle blocking and that PMC integrity was maintained (FIG. 13). 25 G needle was chosen for all the injection purposes.

After injection of NR-labeled PMCs in porcine lumbar discs ex vivo, the fluorescence from NR was visible in the central nucleus pulposus region (the site of injection) for at least 28 days (slightly attenuated fluorescence intensity at later time-points), indicating prolonged retention capability of the PMCs within intervertebral disc tissues.

The retention characteristics of NR-labeled PMCs were also evaluated in canine knee joints in vivo. After intra-articular injection of NR-labeled PMCs, the joint tissues (synovium, femoral condyle, meniscus, patella and tibial plateau) were dissected and then imaged through IVIS instrument. Fluorescence signals were gradually reduced over time, but still observed in all the joint tissues even after 28 days of injection. The maximum retention of NR-labeled PMCs was observed in synovium with minimal attenuation over time. This finding was significant as PMCs retained in the synovium will be able to release their mRNA cargo to adjacent joint tissues. Importantly, the animals did not show any signs of local or systemic toxicity (e.g., weight loss, inflammation or swelling in the joint) acutely or during the observation period of 28 days post injection. These ex vivo and in vivo results conclusively affirm the potential of the fabricated PMCs for prolonged retention within the dynamic tissue environments of intervertebral discs and knee joints, respectively.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A kit comprising:
a) a porous microcarrier prepared from a poly(lactide-co-glycolide) copolymer having a molecular weight of 2,000-100,000 g/mol and a porogen, the porous microcarrier having an average pore size of at least 100 nm; and
b) a composition comprising a nucleic acid-based therapeutic associated with a lipid nanoparticle.

2. The kit of claim 1, wherein the porous microcarrier is in lyophilized form.

3. The kit of claim 1, wherein the porous microcarrier and the composition are separately packaged.

4. The kit of claim 1, wherein the poly(lactide-co-glycolide) copolymer has the structure: 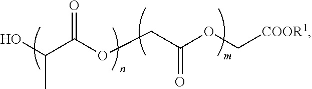
where R¹ is hydrogen or C1-C4 alkyl (ester terminated PLGA), and n and m are independently integers of two or greater.

5. The kit of claim 1, wherein the porogen comprises polyethylene oxide (PEO), polypropylene oxide (PPO), or any combination thereof.

6. The kit of claim 1, wherein the porogen comprises a PEO-PPO-PEO triblock copolymer.

7. The kit of claim 1, wherein the porous microcarrier has an average pore size of 0.5-5 μm.

8. The kit of claim 1, wherein the porous microcarrier has an average particle size of 10-100 μm.

9. The kit of claim 1, wherein the nucleic acid-based therapeutic is RNA, DNA, mRNA, miRNA, siRNA, pDNA, microRNA, ncRNA, engineered msDNA, engineered RT, guide RNA, or a combination thereof.

10. The kit of claim 1, wherein the lipid nanoparticle comprises (a) at least one of a cationic lipid or ionizable lipid, (b) a structural lipid, (c) a sterol, and optionally (d) a polyethylene glycol (PEG)-lipid conjugate.

11. A composition comprising:
a) a porous microcarrier prepared from a poly(lactide-co-glycolide) copolymer having a molecular weight of 2,000-100,000 g/mol and a porogen, the porous microcarrier having an average pore size of at least 100 nm; and
b) a nucleic acid-based therapeutic associated with a lipid nanoparticle.

12. The composition of claim 11, wherein the poly(lactide-co-glycolide) copolymer has the structure: 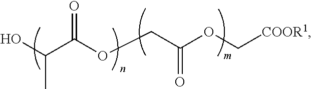
where R¹ is hydrogen or C1-C4 alkyl (ester terminated PLGA), and n and m are independently integers of two or greater.

13. The composition of claim 11, wherein the porogen comprises polyethylene oxide (PEO), polypropylene oxide (PPO), or any combination thereof.

14. The composition of claim 11, wherein the porogen comprises a PEO-PPO-PEO triblock copolymer.

15. The composition of claim 11, wherein the porous microcarrier has an average pore size of 0.5-5 μm.

16. The composition of claim 11, wherein the porous microcarrier has an average particle size of 10-100 μm.

17. The composition of claim 11, wherein the nucleic acid-based therapeutic is RNA, DNA, mRNA, miRNA, siRNA, pDNA, microRNA, ncRNA, engineered msDNA, engineered RT, guide RNA, or a combination thereof.

18. The composition of claim 11, wherein the lipid nanoparticle comprises (a) at least one of a cationic lipid or ionizable lipid, (b) a structural lipid, (c) a sterol, and optionally (d) a polyethylene glycol (PEG)-lipid conjugate.

19. A method of treating a disorder in a subject, comprising administering to the subject the composition of claim 11, wherein the disorder is a musculoskeletal disorder, intervertebral disc degeneration, synovial joint degeneration, mucopolysaccharidoses, arthritis, or a combination thereof.

20. The method of claim 19, wherein administration is parenterally or by injection.
